(19) United States
(12) Patent Application Publication
(10) Pub. No.: US 2006/0167438 A1
(43) Pub. Date: Jul. 27, 2006

(54) Pressure-limiting balloon catheter and method for using the catheter

(76) Inventors: Gary Kalser, Winter Park, FL (US); Gregory L. Mayback, Cooper City, FL (US)

Correspondence Address: CHRISTOPHER & WEISBERG, P.A., 200 EAST LAS OLAS BOULEVARD, SUITE 2040, FORT LAUDERDALE, FL, 33301, US

(21) Appl. No.: 11/339,156

(22) Filed: Jan. 25, 2006

Related U.S. Application Data
(60) Provisional application No. 60/647,204, filed on Jan. 26, 2005.
(60) Provisional application No. 60/647,205, filed on Jan. 26, 2005.

(51) Int. Cl. A61M 27/00

(52) U.S. Cl. 604/544; 604/99.02

(57) ABSTRACT

A pressure-limiting or breakaway catheter includes a multi-lumen shaft having a distal end and a hollow balloon portion disposed at the distal end of the shaft. The balloon portion has a distal end and an interior. The shaft and/or the balloon portion has a balloon safety valve formed to open to the environment outside the shaft and/or the balloon portion when greater than a given bursting pressure exists within the shaft and/or the interior of the balloon portion. The safety valve can be formed to burst at a first breaking force less than a second breaking force required to burst the balloon portion and/or the shaft. Also provided is a breakaway catheter kit including a set of the breakaway catheters, each having the balloon safety valve with different safety valve breaking constants.

### CROSS-REFERENCE TO RELATED APPLICATION

This application claims the benefit under 35 U.S.C. § 119(e) of U.S. Provisional Patent Application Nos. 60/647,204 and 60/647,205, filed Jan. 26, 2005; the prior applications are hereby incorporated herein by reference in their entireties.

### BACKGROUND OF THE INVENTION

1. Field of the Invention

The present invention relates to a catheter, especially a flexible pressure-limiting balloon catheter and a method for using the catheter.

2. Description of Related Prior Art

A number of conventional balloon catheters exist in the prior art. Some catheters are used to drain the bladder of a patient during surgical procedure or to treat bladder and/or urethra or prostate conditions, for example. For example, a common balloon catheter made by RUSCH® and referred to as a Foley catheter is widely used today for treating and draining a patient's bladder. The Foley catheter is shown in FIG. 1 and has a multi-lumen shaft 1 that is disposed in the urethra 10, a balloon portion 3 disposed at the distal end of the shaft 1, a fluid drain section 4 disposed at the distal end of the balloon 3, and a curved, distal guiding tip 5 at the distal-most end of the entire catheter. When placed properly, the proximal-most side of the inflated balloon 3 rests on the interior wall 31 of the bladder 30, entirely blocking off the urethrovesical junction 11 connecting the bladder 30 and the urethra 10. In such a position, the fluid drain section 4 allows continuous drainage of the bladder 30 and the balloon 3 virtually entirely prevents the catheter from slipping out of the bladder. This ideally inserted position is shown in FIG. 1. As used herein, a fluid can be both a liquid and a gas. Exemplary fluids for inflating a balloon 3 are saline, air, or carbon dioxide gas. Exemplary fluids drained by the catheters mentioned herein include urine and blood.

Over 96 million indwelling catheters are sold on an annual basis. Twenty four million catheters are sold to hospitals in the U.S. There are numerous complications associated with those catheters that need to be prevented. These complications are responsible for increases in hospital stays, excessive bleeding, mortality, as well as morbidity. This also causes an increased expense and burden on the already-stressed health care system.

The complications result from several different mechanisms. First, and probably most common, is improper placement of the catheter. Because of the unique anatomy of the male urethra, placing a urethral catheter for urinary drainage can be difficult. A problem arises when the physician, technician, or nurse thinks that the catheter is actually in proper position. The proper position for the catheter is with the balloon being in the cavity of the bladder and the tip distal to the balloon is used to drain the bladder cavity.

For placement of this catheter in the bladder 30 in the ideal position, however, the physician or technician has no visual aid. As shown in FIG. 1, the wall 40 defining the urethrovesical junction 11 is very short in the longitudinal direction of the urethra 10. If the physician inserts the catheter too far into the bladder 30, no damage occurs from balloon inflation; however, there is a possibility of leakage around the balloon 3, which, under normal conditions, helps to lubricate the urethra 10. In such a case, gentle proximal movement will move the proximal side of the balloon 3 against the urethrovesical junction 11. The bladder 30 can then easily expand and stretch to compensate for the balloon 3. A normal bladder capacity is 400 to 500 cc. A normal balloon capacity is approximately 10 to 12 cc although larger balloons are sometimes used.

The complication occurs when the technician and/or nurse inflates the balloon and in fact the balloon is not in the bladder. If the technician does not insert the catheter in far enough, then the balloon 3 will be subsequently inflated within the urethra 10—a condition that is common and, not only is it to be avoided at all costs, is a frequent cause of bladder infections created during a hospital or clinic visit. Infections arise because inflation of the bladder 3 inside the urethra 10 causes the urethra 10 to stretch too far. Even though the urethra 10 is a flexible tube, it has limits to which it can be safely stretched from within. Almost every balloon catheter has an outer diameter/circumference that well-exceeds the safe stretching limit of the urethra 10. Therefore, if the balloon catheter is not inserted far enough, inflation of the balloon 3 will cause serious injury to the urethra 10. This is especially true with elderly patients who have urethra 10 that are not as elastic as younger patients.

In such a case, the balloon expands and tears the surrounding membrane called the mucosa. Tearing of the urethra 10 in this way causes bleeding and allows bacteria to enter into the bloodstream at the tear site, thus causing the subsequent bladder infection. Significant bleeding can become life threatening. The urethra can normally dilate several millimeters; however, when the balloon is inflated, this dilation is usually several centimeters.

Life threatening bleeds, especially in patients who are anticoagulated, can and do occur. Also when the urine is infected, as in immunocompromised patients and the elderly, the bacteria, then, get into the blood stream and can cause serious infections called sepsis, which frequently can lead to death. If the patient survives the initial trauma, then long-term complications, such as strictures, can and usually do occur. Strictures are narrowings within the urine channel and usually require additional procedures and surgeries to correct.

Other mechanisms of catheter-induced injuries are inadvertent manipulation of the tubing or dislodging of the balloon because the balloon is pulled due to a sudden jerk or tension. This usually happens when the patient is ambulating or traveling from the bed to the commode or bathroom. The tubing may inadvertently become fixed while the patient is still moving, at which time a sudden jerk is imparted upon the balloon and pulls the balloon into the urethra causing severe pain and bleeding. Injury caused by the improper, inadvertent, and/or early removal of an inflated balloon catheter is referred to as iatrogenic injury (also referred to as an in-hospital injury).

Yet another scenario occurs when the patient deliberately pulls on the catheter, thereby causing self-induced pain and injury to the urethra. This commonly happens in confused patients, for example, patients in nursing homes who have a disease or cognitive dysfunction problem, such as Alzheimer's disease, or other diseases that make the patient unable to understand the necessity of having a catheter. Confusion occurs when the patient has a spasm causing a strong urge to urinate and pain. During the spasm, the confused patient often tugs and pulls on a catheter, which results in injury.

These types of injuries are not limited to males and also cause severe damage to the female bladder and urethra. The injuries can also occur post-surgically, which makes the damage even more severe. One common situation where injury is cause is when the patient is medicated with morphine or other analgesics that render the patient confused and unable to make rational decisions. These injuries have been well documented and are not limited to adults. Numerous injuries are documented in pediatric patients.

Usually, it takes time to make a diagnosis of patient-caused catheter injury. Immediately after diagnosing the injury, a technician needs to deflate the catheter. However, once the urethra is torn, replacing the damaged catheter with another catheter is quite difficult and, in fact, exacerbates the injury. Sometimes, the patient has to be taken to the operating room to replace a urinary drainage tube once this scenario occurs. Because catheters and leg bags are now used routinely in certain situations during home health care, this scenario is not limited to hospitals and occurs at nursing homes or the patient's home.

Most of the recent catheter technology has been focused on reducing urinary tract infections that are caused by catheters, injuries that are usually the most common catheter-related complications.

A balloon catheter made by TherMatrx, Inc. is used to treat Benign Prostatic Hyperplasia by treatment with THERMATRX® Dose Optimized Thermotherapy. THERMATRX® is a minimally invasive procedure performed in a urologist's office that uses heat delivered through a microwave antenna. A urethral catheter containing the microwave antenna is passed through the urethra and prostate gland and is secured by a balloon at the tip of the catheter that passes through the uretheral sphincter. Localized microwave energy is delivered at a temperature high enough to relieve BPH symptoms, including difficult, frequent, or urgent urination. This catheter is also diagrammatically illustrated in FIG. 1. It differs from the standard Foley catheter by the addition of a radiation coil 2 disposed at a distal end of the shaft 1 proximal to the balloon 3. When placed properly, the radiation coil 2 is immediately adjacent and/or inside the prostate 20 and the proximal-most side of the inflated balloon 3 rests on the interior wall 31 of the bladder 30, entirely blocking off the urethrovesical junction 11. In such a position, the prostate 20 can be directly treated with the radiation coil 2. This ideally inserted position is shown in FIG. 1. Placement of this catheter in the bladder 30 and near the prostrate 20 in the ideal position is the same as the Foley catheter, it is difficult and gives the physician or technician no visual aid. If the balloon 3 of the THERMATRX® catheter is not at the urethrovesical junction 11, then the heating element 2 would not be in the proper position and would heat an undesired area can cause unintended injury.

In a conventional balloon 3, the balloon 3 has a substantially constant balloon wall thickness. The balloon 3 is fixed to the outer surface of a fluid drainage line (not illustrated in FIG. 1) and is not intended to be removed therefrom or to burst thereon unless an extraordinary amount of inflation occurs. If such an event happens, the material of the balloon will open at a random location based upon the microscopic fractures or weaknesses in the material itself. Such a tearing event is not supposed to occur under any circumstances during use with a patient.

Prior art catheters are not constructed to prevent tearing of the urethra during a catheter implanting procedure and are not constructed to break in any predefined way. Accordingly, it would be beneficial to provide a balloon catheter that does not inflate past the tearing limit of a urethra and breaks in a desired, predefined way under certain conditions.

### SUMMARY OF THE INVENTION

It is accordingly an object of the present invention to provide a pressure-limiting balloon catheter and method for using the catheter that overcome the hereinafore-mentioned disadvantages of the heretofore-known devices and methods of this general type and that does not inflate past the tearing limit of a lumen in which the catheter is placed, for example, in the urethra, and quickly and rapidly deflates if pulled out prior to deflation of the balloon.

The purpose of the breakaway catheter of the present invention is to prevent injury by having the balloon automatically deflate before an injury can occur. While the catheter of the present invention makes it a safer device for urinary drainage, the present invention can also be used for any procedures in which balloons are used to dilate cavities. Examples of these procedures include coronary artery vessels, peripheral vascular vessels, such as the aorta and extremity vessels. Balloon dilations of other lumens, such as ureters and the esophagus, are also candidates for use of the catheter of the present invention.

Although deflation of a catheter renders it useless, deflation protects the patient from serious harm. Prevention of such injuries is becoming more and more important because the injuries are commonplace. The increase occurs for a number of reasons. First, a greater percentage of the population is aging. Second, there is a current trend to use less-skilled health care personnel to perform more procedures and to be responsible for treatment, both of which save money. The shortage of nursing professionals (R.N.s) exacerbates this trend. The present tendency is to use nursing professionals for more functions, such as administration and delivery of medications. This leaves only the less-skilled technicians with the task of inserting catheters and taking vital signs. Under such circumstances, more injuries are likely. Lastly, catheter-related complications are becoming more severe due to the increased use of anticoagulation medication, such as Plavix™, that is frequently prescribed in treating cardiovascular disease.

Yet another possible complication arising from the standard Foley catheter is that the balloon will not deflate even when the deflation mechanism is activated. This situation can occur, for example, because the wrong fluid is used to inflate the balloon, such as saline, which can crystallize. Sometimes, the ability to deflate the catheter is interrupted because the drainage channel that is used to deflate the balloon becomes obstructed, which is common if the catheter is left in place too long. Remedy of such a scenario involves an invasive procedure, which includes threading a needle or other sharp object somewhere through the body cavity to puncture the balloon and, thus, dislodge the catheter.

With the breakaway catheter or auto-deflating balloon of the present invention, the technician, nurse, or doctor merely needs to actuate the deflation valve or pull on the catheter to cause the catheter to automatically deflate, thus sparing the patient from any additional surgical procedures.

The added benefit of the present invention is not just for safety, significant financial benefits arise as well.

It is believed that catheter-induced injuries are much more common than public documentation suggests. Catheter-related trauma occurs roughly at least once a week in a large metropolitan hospital. Usually, each incident not only increases the patient's hospital stay substantially, but also the expense of the stay. Each incident (which is usually not reimbursed by insurances) can increase the cost to the hospital thousands, even tens of thousands of dollars.

When additional surgery is required to fix the catheter-induced injury, increased expense to the hospital is not only substantial, if litigation occurs as a result of the injury, damages awarded to the patient can run into the millions of dollars. The catheter of the present invention, therefore, provides a safer catheter that has the possibility of saving the medical industry millions of dollars.

To prevent occurrences of urethra tearing due to premature-improper inflation of the balloon and/or due to premature removal of an inflated balloon, the invention of the instant application provides a balloon safety valve.

The maximum stress that a typical urethra can take without tearing and/or breaking is known and is referred to as a maximum urethra pressure. It is also possible to calculate how much pressure is exerted upon the exterior of a balloon of a balloon catheter by measuring the pressure required to inflate the balloon. Knowing these two values, it is possible to construct a balloon that breaks rapidly and/or ceases inflation if the maximum urethra pressure is exceeded.

For example, in a first embodiment, the balloon, which is typically some kind of rubber, silicone, or plastic, can be made with a breaking point that instantly deflates the balloon if the pressure in the balloon exceeds the maximum urethra pressure. It is acknowledged and accepted that, once the balloon breaks, this catheter is useless and must be discarded because the cost of injury far outweighs the cost of the disposable catheter. Also, such a balloon is limited to inflation with a bio-safe fluid to prevent unwanted air/gas from entering the patient. If, however, air will not injure the patient, the fluid can be air.

As an alternative to a one-use breaking safety valve, a multi-use pressure valve can be added to the balloon inflation lumen and can be set to open into the environment (instead of in the patient) if the maximum urethra pressure exceeded in the balloon or the balloon inflation lumen. Such a valve can be located near or at the balloon inflation port and, in such a configuration, will not enter improperly the patient. Any combination of the above valves is envisioned as well.

With the foregoing and other objects in view, there is provided, in accordance with the invention, a breakaway catheter, including a multi-lumen shaft having a distal end, a hollow balloon portion disposed at the distal end of the shaft, the balloon portion having a distal end and an interior, and at least one of the shaft and the balloon portion having a balloon safety valve formed to open to the environment outside the at least one of the shaft and the balloon portion when greater than a given bursting pressure exists within at least one of the shaft and the interior of the balloon portion.

In accordance with another feature of the invention, the shaft defines a fluid drain lumen and a balloon inflation lumen fluidically connected to the interior of the balloon portion, and the balloon safety valve is formed to open to the environment outside at least one of the shaft and the balloon portion when greater than a given bursting pressure exists within at least one of the balloon inflation lumen and the interior of the balloon portion.

In accordance with a further feature of the invention, the shaft has a proximal end defining a balloon port fluidically connected to the balloon inflation lumen and an inflating connector is fluidically connected to the balloon inflation lumen through the port.

In accordance with an added feature of the invention, the inflating connector is at least a portion of a luer connector.

In accordance with an additional feature of the invention, the proximal end of the shaft defines a fluid port fluidically connected to the fluid drain lumen.

In accordance with yet another feature of the invention, there is provided a fluid drain disposed at the distal end of the balloon portion and fluidically connecting the fluid drain lumen to the environment at the fluid drain and the fluid port to the environment at the proximal end of the shaft.

In accordance with yet a further feature of the invention, the fluid drain has a curved, distal guiding tip.

In accordance with yet an added feature of the invention, the balloon safety valve is at the balloon portion and is fluidically connected to the interior of the balloon portion.

In accordance with yet an additional feature of the invention, the balloon safety valve is integral with the balloon portion.

In accordance with again another feature of the invention, the balloon safety valve is at the shaft and is fluidically connected to the interior of the balloon portion.

In accordance with again a further feature of the invention, the balloon safety valve is integral with the shaft.

In accordance with again an added feature of the invention, the balloon safety valve is removably attached to the shaft.

In accordance with again an additional feature of the invention, the balloon inflation lumen and/or the balloon portion has a material breaking point formed to break and deflate the balloon portion when pressure in at least one of the balloon inflation lumen and the balloon portion exceeds the given pressure.

In accordance with still another feature of the invention, the given pressure is a maximum urethra pressure.

In accordance with still a further feature of the invention, the balloon portion has a wall thickness and the balloon safety valve is a defined reduction in the wall thickness.

In accordance with still an added feature of the invention, the reduction has a shape selected from one of the group consisting of a hemisphere, a cylinder, a groove, a trapezoid, a triangle, a square, a rectangle, a pyramid, and frusto-conical. If the reduction is a groove, then the groove can be formed to extend partly around the balloon portion.

In accordance with still an additional feature of the invention, the shaft defines a balloon inflation lumen fluidically connected to the interior of the balloon portion, the shaft has an outer surface, and the defined reduction is a narrowing of a wall thickness between the balloon portion and the environment and/or the balloon inflation lumen and the outer surface of the shaft.

In accordance with another feature of the invention, the balloon inflation lumen and the outer surface define a wall therebetween, and the defined reduction is a narrowing of a thickness of the wall from the outer surface towards the balloon inflation lumen and/or from the balloon inflation lumen towards the outer surface.

In accordance with a further feature of the invention, the shaft has a wall thickness and the balloon safety valve is a defined reduction in the wall thickness.

In accordance with an added feature of the invention, the balloon safety valve opens into the environment and/or a patient when broken.

In accordance with an additional feature of the invention, the balloon safety valve is formed to burst at a first breaking force less than a second breaking force required to burst at least one of the balloon portion and the shaft.

In accordance with yet another feature of the invention, at least one of the balloon portion and the shaft is formed to burst at a first breaking force and the balloon safety valve is formed to burst at a second breaking force less than the first breaking force.

With the objects of the invention in view, there is also provided a breakaway catheter, including a multi-lumen shaft having a distal end, a hollow balloon portion disposed at the distal end of the shaft, the balloon portion having a distal end and an interior, and at least one of the shaft and the balloon portion having a balloon safety valve formed to burst at a first breaking force less than a second breaking force required to burst at least one of the balloon portion and the shaft.

With the objects of the invention in view, there is also provided a pressure-limiting balloon catheter, including a multi-lumen shaft having first and second lumen each shaped to pass a respective fluid therethrough, a hollow balloon portion defining an interior chamber fluidically connected to the first lumen for receiving fluid therein to inflate and deflate at least a part of the balloon portion, and a pressure-limiting valve fluidically connected to at least one of the balloon portion and the first lumen, the valve opening to the environment of at least one of the shaft and the balloon portion when greater than a given bursting pressure exists in the least one of the balloon portion and the first lumen.

With the objects of the invention in view, there is also provided a pressure-limiting balloon catheter, including a multi-lumen shaft having a balloon inflation lumen shaped to pass a balloon-inflating fluid therethrough and a catheter lumen shaped to pass a fluid therethrough, a hollow balloon portion defining an interior chamber fluidically connected to the balloon inflation lumen for receiving the balloon-inflating fluid therein to inflate and deflate at least a part of the balloon portion, and a pressure-limiting valve fluidically connected to at least one of the balloon portion and the balloon inflation lumen, the valve opening to the environment of at least one of the shaft and the balloon portion when greater than a given bursting pressure exists in the least one of the balloon portion and the balloon inflation lumen.

With the objects of the invention in view, there is also provided a breakaway catheter kit, including a set of breakaway catheters according to the present invention, each of the catheters having the balloon safety valve with different safety valve breaking constants.

Other features that are considered as characteristic for the invention are set forth in the appended claims.

Although the invention is illustrated and described herein as embodied in a pressure-limiting balloon catheter and method for using the catheter, it is, nevertheless, not intended to be limited to the details shown because various modifications and structural changes may be made therein without departing from the spirit of the invention and within the scope and range of equivalents of the claims.

The construction and method of operation of the invention, however, together with additional objects and advantages thereof, will be best understood from the following description of specific embodiments when read in connection with the accompanying drawings.

### BRIEF DESCRIPTION OF THE DRAWINGS

FIG. 1 is a longitudinally diagrammatic cross-sectional view of a prior art catheter ideally placed in a urethra and a bladder of a male patient;

FIG. 2 is a fragmentary, enlarged, cross-sectional view of a distal portion of a first embodiment of a pressure-limiting balloon catheter according to the invention;

FIG. 3 is a fragmentary, enlarged cross-sectional view of a proximal portion of a second embodiment of a pressure-limiting balloon catheter according to the invention;

FIG. 4 is a fragmentary, enlarged, cross-sectional view of a first alternative configuration of the safety valve of FIG. 3;

FIG. 5 is a fragmentary, enlarged, cross-sectional view of a second alternative configuration of the safety valve of FIG. 3;

FIG. 6 is a fragmentary, enlarged, cross-sectional view of a third alternative configuration of the safety valve of FIG. 3;

FIG. 7 is a fragmentary, further enlarged, cross-sectional view of the safety valve of FIG. 6;

FIG. 8 is a fragmentary, further enlarged, cross-sectional view of a fourth alternative configuration of the safety valve of FIG. 3; and

FIG. 9 is a fragmentary, perspective view of a fifth alternative configuration of a retrofitting safety valve according to the invention.

### DETAILED DESCRIPTION OF THE PREFERRED EMBODIMENTS

While the specification concludes with claims defining the features of the invention that are regarded as novel, it is believed that the invention will be better understood from a consideration of the following description in conjunction with the drawing figures, in which like reference numerals are carried forward.

Before the present invention is disclosed and described, it is to be understood that the terminology used herein is for the purpose of describing particular embodiments only and is not intended to be limiting. It must be noted that, as used in the specification and the appended claims, the singular forms “a,” “an,” and “the” include plural references unless the context clearly dictates otherwise.

Referring now to the figures of the drawings in detail and first, particularly to FIG. 2 thereof, there is shown a first embodiment of a pressure-limiting balloon catheter 100 that does not inflate past the tearing limit of a lumen in which the catheter 100 is placed, for example, in the urethra.

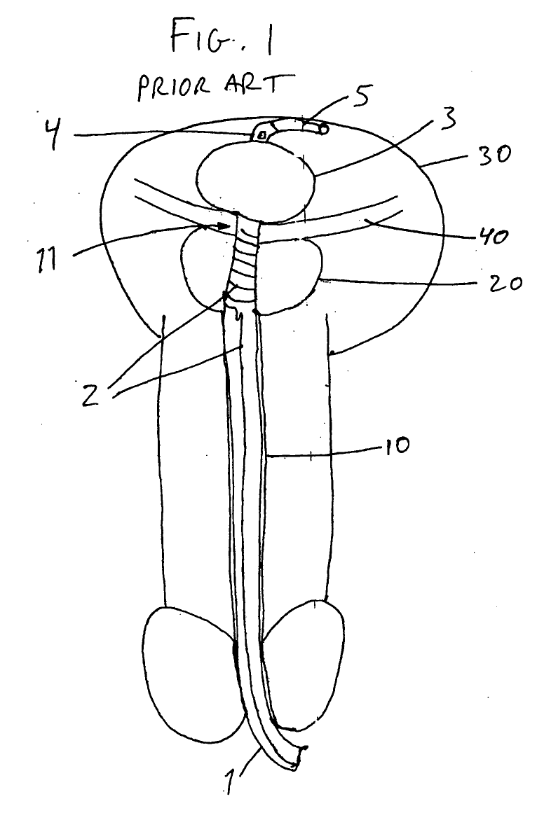

To prevent occurrences of urethra tearing due to premature-improper inflation of the balloon and/or due to premature removal of an inflated balloon, the invention of the instant application provides the balloon 110 with a balloon safety valve 112. As set forth above, in a balloon 3 of a conventional catheter (see reference numerals 1 to 5 in FIG. 1), the balloon 3 is fixed to the outer surface of the fluid drainage line 120 (not shown in FIG. 1) and is not intended to be removed therefrom or to burst thereon unless an extraordinary amount of inflation occurs. Such a tearing event is not supposed to occur under any circumstances during use with a patient. If such an event happens, the material of the balloon 3 will open at a random location, based upon the microscopic fractures or weaknesses in the material itself, and risk of serious damage to the patient is associated with the bursting, as well as a risk of balloon fragmentation, which could leave pieces of the balloon 3 inside the patient after removal of the catheter.

In contrast to such conventional devices, the balloon 110 of the present invention is created specifically to tear when a predefined pressure exists in or is exerted on the balloon 110. The controlled tear will occur because the balloon safety valve 112 is present. Conventional balloons have constant balloon wall thicknesses. In contrast thereto, the balloon safety valve 112 in the first embodiment is a defined reduction in balloon wall thickness. This reduction creates a breaking point or selected breaking points at which the balloon 110 is intended specifically to break when a predefined force exists in or is imparted on the balloon 110. Because the balloon 110 is made of a material having a known tearing constant—dependent upon the thickness thereof (which is determined experimentally for different thicknesses of a given material prior to use in a patient), the balloon safety valve 112 of the present invention for urethra applications is matched to break when the pressure inside or exerted on the balloon 110 approaches the maximum urethra pressure.

In the embodiment shown in FIG. 2, a decreased thickness is formed as a first semi-circumferential groove 114 near a proximal end of the balloon 110 and/or as a second semi-circumferential groove 116 near a distal end of the balloon 110. The grooves 114, 116 can have any cross-sectional shape, including, trapezoidal, triangular, square, or rectangle, for example. Because rubber, plastic, and silicone materials tear well with thinner cuts, a relatively triangular shape or one with a narrow bottom is preferred. To make sure that the entire balloon 110 of the illustrated embodiment does not completely tear away from the fluid drainage line 120, both grooves 114, 116 do not extend around the entire circumference of the balloon 110. As shown to the left of the proximal groove 116 in FIG. 2, the groove 116 is not present on at least an arc portion 118 of the circumference of the balloon 110. The arc portion is defined to be sufficiently large so that, when the catheter 100 is removed from the patient, the balloon 110 cannot tear away entirely from the catheter 100 (and create the disadvantageous fragmentation situation as set forth above). The illustrated balloon safety valve 112 is, therefore, fashioned to keep the balloon 110 in one piece after breaking and firmly connected to the catheter 100 to insure that no piece of the balloon 110 will be left inside the patient after actuation of the balloon safety valve 112.

It is noted that the balloon 110 is inflated through an inflation lumen 130 having a proximal opening, typically formed by a female end of a luer connector. The female end is connected to a non-illustrated inflation device, for example, a distal end of a syringe for inflation of the balloon 110.

In this first embodiment, the balloon can be of rubber, silicone, or plastic, for example. Once the balloon breaks, the catheter is useless and must be discarded. Because the balloon 110 in this embodiment will break inside the patient, it should be inflated with a bio-safe fluid to prevent an unwanted air or gas from entering the patient. In certain circumstances where balloon catheters are used, air or gas will not injure the patient if let out into the patient's body cavity. In such circumstances, the inflating fluid can be air under pressure, for example.

Maximum urethra pressure can also be tailored to the individual patient. Based upon a urethral pressure-measuring device, the patient's maximum urethra pressure can be measured before the catheter 100 is placed therein. A set of catheters 100 having different safety valve breaking constants can be available to the physician and, after estimating or calculating or knowing the patient's maximum urethra pressure, the physician can select the catheter 100 having a safety valve breaking constant slightly or substantially smaller than the patient's maximum urethra pressure. Accordingly, if the pressure in the balloon 110 approaches the patient's maximum urethra pressure for any reason, whether it is due to over-inflation, improper placement, and/or premature removal, the balloon 110 is guaranteed to break prior to the patient's lumen, in particular, the patient's urethra, prior to causing iatrogenic injury.

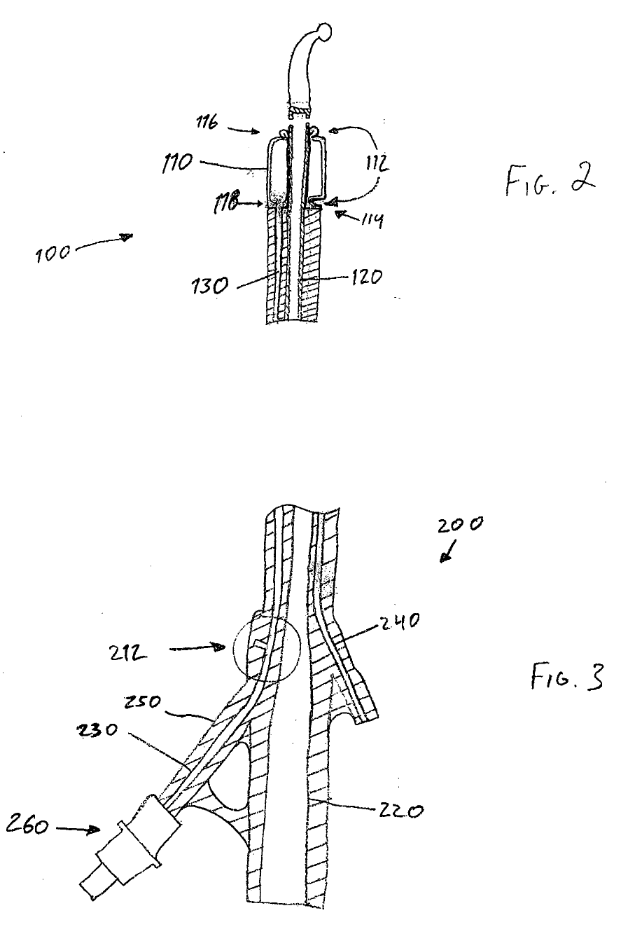

A second embodiment of the one-use breaking safety valve of a pressure-limiting balloon catheter 200 is shown in FIG. 3. The catheter 200 has a fluid drainage line 220, a balloon inflation lumen 230, and a secondary lumen 240.

The fluid drainage line 220 is connected fluidically to the body cavity (i.e., the bladder 30) for draining fluid from the body cavity.

The secondary lumen 240 can be used for any purpose, for example, for housing the radiation line that will supply energy to the radiation coil 2. It can also be used for injecting fluid into any distal part of the catheter 200 or even the body cavity itself.

The balloon inflation lumen 230 begins at a proximal end with an inflating connector 260 that, in a preferred embodiment, is a female luer connector (of course, it can be a male luer connector too). The balloon inflation lumen 230 continues through the body of the catheter 200 all the way to the balloon and is fluidically connected to the interior of the balloon.

The balloon safety valve is also fluidically connected to the balloon inflation lumen 230. In the second embodiment of the safety valve 212, the valve 212 is formed integrally with the balloon inflation lumen 230 and is set to open into the environment (instead of into the patient) if the maximum urethra pressure is exceeded in the balloon or the balloon inflation lumen. Because this safety valve 212 is located near or at the balloon inflation port 220 in this configuration, fluid used to inflate the balloon will not enter the patient when the valve 212 opens.

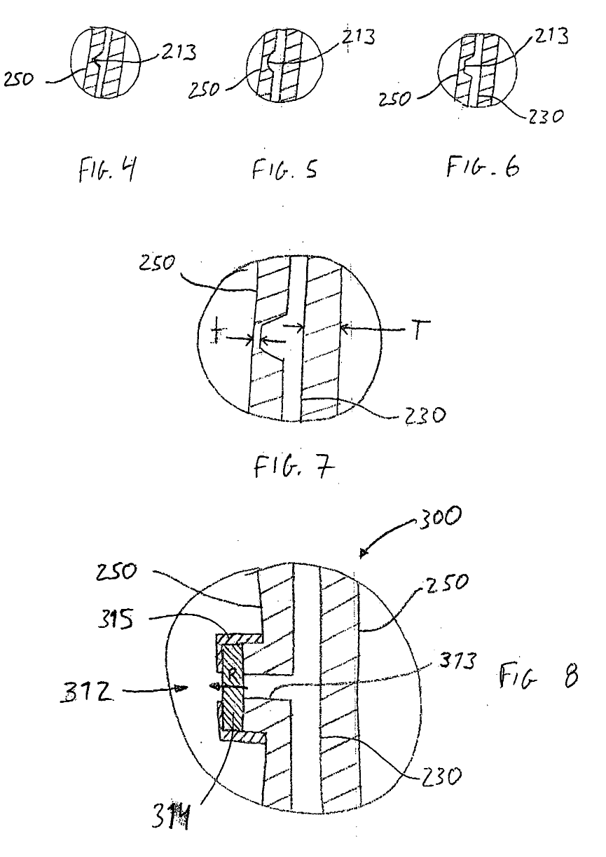

The safety valve 212 in the second embodiment can merely be a narrowing of the distance between the balloon inflation lumen 230 and the outer surface 250 of the catheter 220. In FIG. 3, the valve 212 has a rectangular cross-section and extends away from the balloon inflation lumen 230. As shown in FIGS. 4, 5, and 6, respectively, the cross-section can be triangular (peaked or pyramidical in three-dimensions), curved (circular or cylindrical in three-dimensions), or trapezoidal (frusto-conical or bar-shaped in three-dimensions). The cross-sections are shown in FIGS. 3 to 7 with the narrowing emanating from the balloon inflation lumen 230 outward. As an alternative, the narrowing can begin on the outer surface of the catheter and extend inwards towards the balloon inflation lumen 230. A further alternative can have the narrowing extend from both the lumen 230 and the outer surface of the catheter.

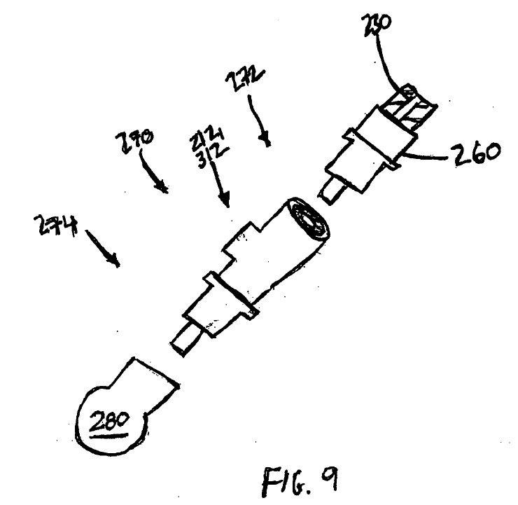

The cross-sections illustrated are merely exemplary. What is important is that the thickness t between the bottom 213 of the valve 212 and the outer surface 250 of the catheter 220 in comparison to the thickness T of the catheter body over the remainder of the balloon inflation lumen 230. An enlarged view of this thickness comparison is illustrated in FIG. 7. As long as the thickness t is smaller than the thickness T (t<T), and as long as the force F_{b} required to break the balloon is greater than the force Fₛᵥ required to break the portion 213 of the safety valve 212 (F_{b}>Fₛᵥ), then the portion 213 of the safety valve 212 is virtually guaranteed to break every time pressure exerting a force F in the balloon inflation lumen 230 is greater than the force Fₛᵥ required to break the safety valve (Fₛᵥ>F)

Based upon this analysis, the force Fₛᵥ required to break the safety valve can be tuned to whatever a patient needs or a physician desires and different sized valves can be available for any procedure and provided in the form of a kit. Whether a standard maximum urethra pressure is used or a patient-specific maximum urethra pressure is measured and used, experiments can be conducted prior to use on a patient on various catheter thicknesses t to determine the pressure needed to break the portion 213 of the safety valve 212. For example, ten different maximum urethra pressures can be known as desirable setpoints and the thicknesses t can be varied such that pressure required to break the ten thicknesses correspond to the ten setpoint pressures. If, then, ten catheters are placed in such a kit, each having one of the ten thicknesses, then the physician has a range of 10 maximum urethra pressure values to use with the patient.

The safety valve 212 of the second embodiment need not be confined to the body of the catheter 200. Instead, the inflating connector 260 can be equipped with the safety valve 212. Alternatively, a modular attachment 270 containing the safety valve 212 can be attached to the inflating connector 260. Such a modular valve attachment 270 is removable and replaceable (such as through a convention luer or even a screw-threaded connection). Accordingly, as long as the catheter 200 can still be used after the valve 212 actuates (breaks), the used attachment 270 can be replaced with a new attachment 270. The converse is also true for reuse of the attachment 270 if the catheter 200 breaks and the valve of the attachment 270 remains unbroken.

One embodiment of the attachment is illustrated in FIG. 9. Specifically, an upstream end of the connector 260 is attached removably to a downstream end 272 of the modular valve attachment 270 and the upstream end 274 of the attachment 270 is attached to the female connection of the balloon inflation device 280 illustrated only diagrammatically in FIG. 9. A common exemplary inflation device 280 is a syringe.

In such a configuration, the safety valve 212, 312 of the present invention can be entirely separate from the catheter 200, 300 and, therefore, form a retrofitting device for attachment to the luer connector present on conventional catheters.

As an alternative to the one-use breaking safety valve of the second embodiment, a multi-use pressure valve can be used. This third embodiment of the pressure-limiting balloon catheter 300 is illustrated in FIG. 8. The catheter 300 can be the same as the catheter 200 in FIG. 3 except for the portion illustrated in FIG. 8. Instead of having a narrowing thickness t of the lumen wall, the valve portion 313 extends entirely to the environment. However, a one-way valve 314 (shown only diagrammatically in FIG. 8) is attached to the open end of the valve portion 313 and is secured to the outer surface 250 of the catheter 300 to close off the open end of the valve portion 313. The one-way valve 314 can be secured directly to the outer surface 250 (e.g., with an adhesive) or a connector 315 (e.g., a threaded cap) can secure the one-way valve 314 to the open end of the valve portion 313. Regardless of the configuration, the one-way valve 314 includes a device that does not permit fluid from exiting the lumen 230 until a given resistance R is overcome. This given resistance R can be selectable by the physician depending upon the one-way valve that is chosen for use if a set of one-way valves having different resistances R are available for use by the physician. Just like the second embodiment, the resistance R can be set to correspond to desired maximum urethra pressure values. Therefore, when used, the fluid exits the one-way valve 314 into the environment well before the patient's maximum urethra pressure is exceeded by the balloon.

The one-way valve 314 can be a mechanical one-way valve. Additionally, the one-way valve 314 can be a material having a tear strength corresponding to the desired set of resistances R. The material can be a fluid-tight fabric, a rubber, a plastic, or silicone different from the material making up the catheter. The material can even be a rubber, plastic, or silicone the same as the material making up the catheter but having a reduced thickness t than the thickness T of the catheter.

Because the safety valve 212, 312 is located at the proximal end of the catheter 200, 300, the distal end of the catheter 200, 300 can take the form of a distal end of a conventional balloon catheter 2, 3, 4, 5. Alternatively, the distal end shown in FIG. 2 can also be used for redundant over-pressure protection.

The catheter 200, 300 according to the invention can be used in vascular applications. It is known that every vessel has a tearing pressure. Balloons are used in coronary arteries, for example. If a coronary artery balloon were to burst, there would be less damage if the burst was controlled according to the invention. The same is true for a renal or iliac blood vessel. In such situations, the breakaway catheter improves upon existing catheters by making them safer. From the urinary standpoint, the breakaway balloon will not only prevent injury, but will also be a signal to the technician that he/she needs to obtain the assistance of a physician or urologist with respect to inserting the catheter.

We claim:

1. A breakaway catheter, comprising:
a multi-lumen shaft having a distal end; and
a hollow balloon portion disposed at said distal end of said shaft, said balloon portion having a distal end and an interior; and
at least one of said shaft and said balloon portion having a balloon safety valve formed to open to the environment outside said at least one of said shaft and said balloon portion when greater than a given bursting pressure exists within at least one of said shaft and said interior of said balloon portion.

2. The catheter according to claim 1, wherein said shaft defines:
a fluid drain lumen; and
a balloon inflation lumen fluidically connected to said interior of said balloon portion; and
said balloon safety valve is formed to open to the environment outside at least one of said shaft and said balloon portion when greater than a given bursting pressure exists within at least one of said balloon inflation lumen and said interior of said balloon portion.

3. The catheter according to claim 2, wherein:
said shaft has a proximal end defining a balloon port fluidically connected to said balloon inflation lumen; and
an inflating connector is fluidically connected to said balloon inflation lumen through said port.

4. The catheter according to claim 3, wherein said inflating connector is at least a portion of a luer connector.

5. The catheter according to claim 3, wherein said proximal end of said shaft defines a fluid port fluidically connected to said fluid drain lumen.

6. The catheter according to claim 5, further comprising a fluid drain disposed at said distal end of said balloon portion and fluidically connecting:
said fluid drain lumen to the environment at said fluid drain; and
said fluid port to the environment at said proximal end of said shaft.

7. The catheter according to claim 6, wherein said fluid drain has a curved, distal guiding tip.

8. The catheter according to claim 1, wherein said balloon safety valve is at said balloon portion and is fluidically connected to said interior of said balloon portion.

9. The catheter according to claim 8, wherein said balloon safety valve is integral with said balloon portion.

10. The catheter according to claim 1, wherein said balloon safety valve is integral with said balloon portion.

11. The catheter according to claim 1, wherein said balloon safety valve is at said shaft and is fluidically connected to said interior of said balloon portion.

12. The catheter according to claim 11, wherein said balloon safety valve is integral with said shaft.

13. The catheter according to claim 11, wherein said balloon safety valve is removably attached to said shaft.

14. The catheter according to claim 2, wherein at least one of said balloon inflation lumen and said balloon portion has a material breaking point formed to break and deflate said balloon portion when pressure in at least one of said balloon inflation lumen and said balloon portion exceeds said given pressure.

15. The catheter according to claim 2, wherein said balloon inflation lumen has a material breaking point formed to break and deflate said balloon portion when pressure in at least one of said balloon inflation lumen and said balloon portion exceeds said given pressure.

16. The catheter according to claim 2, wherein said balloon portion has a material breaking point formed to break and deflate said balloon portion when pressure in at least one of said balloon inflation lumen and said balloon portion exceeds said given pressure.

17. The catheter according to claim 1, wherein said given pressure is a maximum urethra pressure.

18. The catheter according to claim 1, wherein:
said balloon portion has a wall thickness; and
said balloon safety valve is a defined reduction in said wall thickness.

19. The catheter according to claim 18, wherein said reduction has a shape selected from one of the group consisting of a hemisphere, a cylinder, a groove, a trapezoid, a triangle, a square, a rectangle, a pyramid, and frusto-conical.

20. The catheter according to claim 19, wherein said reduction is a groove extending partly around said balloon portion.

21. The catheter according to claim 18, wherein:
said shaft defines a balloon inflation lumen fluidically connected to said interior of said balloon portion;
said shaft has an outer surface; and
said defined reduction is a narrowing of a wall thickness between at least one of:
said balloon portion and the environment; and
said balloon inflation lumen and said outer surface of said shaft.

22. The catheter according to claim 21, wherein:
said balloon inflation lumen and said outer surface define a wall therebetween; and
said defined reduction is a narrowing of a thickness of said wall at least one of:
from said outer surface towards said balloon inflation lumen; and
from said balloon inflation lumen towards said outer surface.

23. The catheter according to claim 1, wherein:
said shaft has a wall thickness; and
said balloon safety valve is a defined reduction in said wall thickness.

24. The catheter according to claim 1, wherein said balloon safety valve opens into the environment when broken.

25. The catheter according to claim 1, wherein said balloon safety valve opens into a patient when broken.

26. The catheter according to claim 1, wherein said balloon safety valve is formed to burst at a first breaking force less than a second breaking force required to burst at least one of said balloon portion and said shaft.

27. The catheter according to claim 1, wherein:
at least one of said balloon portion and said shaft is formed to burst at a first breaking force; and
said balloon safety valve is formed to burst at a second breaking force less than said first breaking force.

28. A breakaway catheter, comprising:
a multi-lumen shaft having a distal end;
a hollow balloon portion disposed at said distal end of said shaft, said balloon portion having a distal end and an interior; and
at least one of said shaft and said balloon portion having a balloon safety valve formed to burst at a first breaking force less than a second breaking force required to burst at least one of said balloon portion and said shaft.

29. A breakaway catheter kit, comprising:
a set of breakaway catheters according to claim 1, each of said catheters having said balloon safety valve with different safety valve breaking constants.

30. A pressure-limiting balloon catheter, comprising:
a multi-lumen shaft having first and second lumen each shaped to pass a respective fluid therethrough;
a hollow balloon portion defining an interior chamber fluidically connected to said first lumen for receiving fluid therein to inflate and deflate at least a part of said balloon portion; and
a pressure-limiting valve fluidically connected to at least one of said balloon portion and said first lumen, said valve opening to the environment of at least one of said shaft and said balloon portion when greater than a given bursting pressure exists in said least one of said balloon portion and said first lumen.

31. A pressure-limiting balloon catheter, comprising:
a multi-lumen shaft having:
a balloon inflation lumen shaped to pass a balloon-inflating fluid therethrough; and
a catheter lumen shaped to pass a fluid therethrough;
a hollow balloon portion defining an interior chamber fluidically connected to said balloon inflation lumen for receiving the balloon-inflating fluid therein to inflate and deflate at least a part of said balloon portion; and
a pressure-limiting valve fluidically connected to at least one of said balloon portion and said balloon inflation lumen, said valve opening to the environment of at least one of said shaft and said balloon portion when greater than a given bursting pressure exists in said least one of said balloon portion and said balloon inflation lumen.
